(19) United States
(12) Patent Application Publication
(10) Pub. No.: US 2025/0230390 A1
(43) Pub. Date: Jul. 17, 2025

(54) MICROFLUIDIC AND PERFUSION BASED CELL-PATTERNING FOR DEVELOPMENT OF CARDIAC ORGANOIDS

(71) Applicant: Board of Regents, The University of Texas System, Austin, TX (US)

(72) Inventors: Binata Joddar, El Paso, TX (US); Sylvia Lucia Natividad-Diaz, El Paso, TX (US); Aibhlin A. Esparza, Austin, TX (US); Naveen Nagiah, Austin, TX (US)

(21) Appl. No.: 18/410,859

(22) Filed: Jan. 11, 2024

(51) Int. Cl. C12M 3/00; C12M 1/00; C12M 3/04; C12M 3/06

(52) U.S. Cl. CPC C12M 21/08; C12M 23/16; C12M 23/38; C12M 27/10; C12M 29/10

(57) ABSTRACT

Embodiments of the invention include additional methods and compositions for fabricating organoids. A unique approach has been developed for the rapid aggregation of defined number of cells using a micropillar-based template to limit the size of organoids, which are then more commonly referred to as spheroids. This procedure can be utilized to create in vitro cardiac tissue models (e.g., cardiac organoids) simply, rapidly, and inexpensively. In certain aspects an alginate-gelatin based hydrogel premixed with cardiomyocytes is used in combination with a patterned microfluidic chip/cell culture chamber having an array of micropillars to fabricate 3D cardiac organoids.

### STATEMENT AS TO RIGHTS TO INVENTIONS MADE UNDER FEDERALLY-SPONSORED RESEARCH OR DEVELOPMENT

This invention was made with government support under grant 1927628 awarded by the National Science Foundation. The government has certain rights in the invention.

### FIELD

Embodiments of the invention are directed generally to cellular biology and in vitro culture, and particularly to formation and use of organoids.

### BACKGROUND OF THE INVENTION

Although organoids have gained significant attention in the pharmaceutical industry, there remain major challenges that prevent such models from achieving broader utilization. One of the key limitations in using organoid-based approaches to generate functional tissue is that upon reaching a certain size, organoids cease to proliferate and develop a necrotic core. The process of growth arrest is thought to be linked to the loss of cell viability in the inner core of the organoid and subsequent necrosis upon reaching a limiting size beyond which diffusion alone can no longer allow for oxygen, nutrient and metabolite exchange. There is a need for additional methods for producing organoids.

### BRIEF SUMMARY OF THE INVENTION

As a solution to the problems detailed above additional methods and compositions for fabricating organoids have been developed. The rapid aggregation of defined number of cells using a micropillar-based template is used to limit the size of organoids, which are then more commonly referred to as spheroids. This procedure can be utilized to create in vitro cardiac tissue models (e.g., cardiac organoids) simply, rapidly, and inexpensively. In certain aspects a hydrogel premixed with cardiomyocytes is used in combination with a patterned microfluidic chip/cell culture chamber having an array of micropillars as a support to fabricate 3D cardiac organoids/spheroids (the micropillars having for example a diameter of 100, 200, 300, 400, 500, 600, 700, 800, 900 to 1000 μm, including any value or range there between; a height of 100, 200, 300, 400, 500, 600, 700, 800, 900 to 1000 μm, including all values and ranges there between; and a spacing of 200, 300, 400, to 500 μm between pillars). The micropillar dimension can provide a micropillar with a top or formation surface having a diameter of 100, 200, 300, 400, 500, 600, 700, 800, 900 to 1000 μm, including any value or range there between. The term “hydrogel” refers to a water-containing three-dimensional hydrophilic polymer network or gel in which the water is the continuous phase, e.g., an alginate-gelatin hydrogel. The hydrogel can comprise of 2 to 80 weight percent alginate or other polysaccharide. In certain aspects the microfluidic chip and/or culture chamber can be fabricated with 3D Stereolithography printing (3D SLA). The term micropillar as used herein refers to a structural element of a cell culture chamber or microfluidic chip and is a column extending from the base of a cell culture chamber, the micropillars having a top surface on which organoids are formed. A micropillar can have a circular or polygonal cross section. In certain aspects the micropillars have a circular cross section. The micropillars in the microfluidic chip or the cell culture chamber serve as inductive templates for organoid assembly when the entire microfluidic chip containing a cell-gel mixture is subjected to rotation and perfusion, for example with a Synthecon Bioreactor (Rotating Wall Perfusion Bioreactor, NASA), under appropriate culture conditions in vitro (e.g., 43 rpm, 5% CO₂, 37° C.). Upon seeding, cardiomyocytes aligned and clustered atop the micropillars along the direction of the fluid flow. After culture, the clustered and aligned myocytes form dense cardiac organoids.

Certain embodiments are directed to a cardiac organoid platform comprising an induction support and an induction media, (i) the induction support comprising micropillars extending from the base of the support and (ii) the induction media comprising a cardiomyocyte and/or cardiomyocyte precursor and a hydrogel support. In certain aspects the micropillars are 0.1 mm to 1 mm in height and 100 μm to 1 mm in diameter. The micropillars can be spaced at a distance of at least or at most 0.05 mm to 0.1 mm or at a density of 100 to 10,000 pillars per mm². In certain aspects the hydrogel is a polysaccharide hydrogel. In particular aspects the polysaccharide is alginate. The alginate hydrogel can be an alginate-gelatin hydrogel. In certain aspects the alginate hydrogel comprises 2 to 80 wt % alginate. The cardiomyocyte can be a pluripotent stem cell-derived cardiomyocyte. In certain aspects the pluripotent stem cell-derived cardiomyocyte is a patient-specific pluripotent stem cell-derived cardiomyocyte. The induction support can comprise a base with an array of micropillars and a lid, the lid and base forming a cell culture chamber when in use.

Other embodiments are directed to a cardiac organoid culture system comprising the cardiac organoid platform described above and a rotation mechanism configured to rotate the cardiac organoid platform during culture.

Certain embodiments are directed to methods of inducing organoid formation comprising: encapsulating an induction support having an array of micropillars forming an encapsulated induction support; culturing the encapsulated induction support in a cell culture chamber while rotating and perfusing the encapsulated induction template. The encapsulated induction support can be cultured in a culture medium at a temperature of 35 to 40° C. and 2 to 8% CO₂ atmosphere. In certain embodiments the encapsulated induction support is rotated at 20 to 60 rpms during culture.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention. Furthermore, compositions and kits of the invention can be used to achieve methods of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1C are schematic illustrations of: (FIG. 1A) Top view of the first layer (lid), (FIG. 1B) Top view of the second layer (chip or cell chamber base), (FIG. 1C) Top and side view of both layers assembled.

FIGS. 2A-2D illustrate (FIG. 2A) a top view, (FIG. 2B) a cross-section, (FIG. 2C) a perspective view and (FIG. 2D) perspective view cross-section illustrating the micropillars of the second layer or cell chamber base.

FIGS. 3A-3B illustrate a perspective views of assembly (FIG. 3A) non-pillared cell chamber and assembly (FIG. 3B) pillared cell chamber.

FIG. 4A illustrates three-dimensional fluorescence images of results of static cell and gel mixture culture.

FIG. 4B illustrates three dimensional fluorescence images of results of a dynamic cell and gel mixture culture.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure or the claims are limited to that embodiment.

Cardiac organoids are 3-dimensional (3D) structures composed of tissue or niche-specific cells encapsulated in either a naturally derived or synthetic extracellular matrix scaffold and may include exogenous biochemical signals such as essential growth factors. The overarching goal of developing cardiac organoid models is to establish a functional integration of cardiomyocytes with physiologically relevant cells, tissues, and structures such as capillary-like networks composed of endothelial cells. These organoids can be used to model human heart anatomy, physiology, and disease pathologies in vitro and have the potential to solve many issues related to cardiovascular drug discovery and can be used in fundamental research. The advent of patient-specific human-induced pluripotent stem cell-derived cardiovascular cells provide a unique, single-source approach to study the complex process of cardiovascular disease progression through organoid formation and incorporation into relevant, controlled microenvironments such as microfluidic devices. Strategies that aim to accomplish such a feat include microfluidic technology-based approaches, microphysiological systems, microwells, microarray-based platforms, 3D bioprinted models, and electrospun fiber mat-based scaffolds. The engineering or technology-driven practices for making cardiac organoid models in comparison with self-assembled or scaffold-free methods to generate organoids are described herein.

There is significant interest in in vitro cardiac organoid and tissue-on-a-chip models for cardiovascular drug discovery and research because they provide inexpensive, controlled, and reproducible platform for better quantification of isolated cellular processes in response to a biochemical or biophysical stimulus. Cardiac organoids can model human heart structure, physiology, and disease pathologies in vitro.

Embodiments of the invention use a microfluidics-based approach to develop a high-throughput bioengineered human cardiac organoid platform, which provides functional contractile tissue with biological properties similar to native heart tissue. A representative micropillar-based design was used to assemble 3D cardiac organoids using, for example but not limited to a 3D Stereolithography (SLA) printed platform and cardiac cells including human AC16 cells as a model system. AC16 cells are a proliferating human cardiomyocyte cell line that was derived from the fusion of primary cells from adult human ventricular heart tissues with SV40 transformed, uridine auxotroph human fibroblasts. AC16 can be serially passaged and can differentiate when cultured in mitogen-free medium. AC-16 can be used to address questions of cardiac biology at the cellular and molecular levels. Typically, AC-16 cells are cultured in DMEM/F12 (Sigma Cat. No. D6434) containing 2 mM L-Glutamine (EMD Millipore Cat. No. TMS-002-C), 12.5% FBS (EMD Millipore Cat. No. ES-009-B) and 1X Penicillin-Streptomycin Solution (EMD Millipore Cat. No. TMS-AB2-C) or an equivalent culture medium.

The cells can include cardiomyocytes or cardiomyocyte precursors such as pluripotent stem cell (PSCs). As an alternative, PSC can be replaced by omnipotent or totipotent stem cells. Accordingly, in the process the stem cells can optionally be at least pluripotent stem cells. Generally preferred, the PSC are generated without use of a human embryo. Preferably, PSC are human PSC (hPCS), e.g. induced PSC (iPSC), e.g. generated from a mammalian cell sample, e.g. a blood or tissue sample, especially a human induced PSC (hiPSC), or an embryonic stem cell line (ESC), which preferably is non-human, or a human embryonic stem cell line (hESC). Generally, the PSC or ESC are not generated using a human embryo. Other cell types can be included as well. The pluripotent stem cells can originate from a patient, e.g. from a tissue sample taken from a patient. The patient can be diagnosed with a disease or can be suspected of having a disease, and the process for producing the cardiac organoids can be used to analyze the effect of a test compound onto the cardiac organoids, e.g. during or following their development to a pre-determined size, wherein the test compound can be a pharmaceutical compound for use in the treatment of the disease. Such a process can be used to analyze the effect of the pharmaceutical compound prior to administration of the compound to the patient.

The PSCs or cardiomyocytes can be cultured with other non-myocyte cell types. In some embodiments, the cardiomyocytes may be cultured with the non-myocytes at a ratio of about 95:5 to about 5:95 of cardiomyocytes to non-myocytes. In some embodiments, the cardiomyocytes may be cultured with the non-myocytes at a ratio is about 60:40 to about 40:60 of cardiomyocytes to non-myocytes. In some embodiments, the non-myocytes may comprise fibroblasts (FBs), endothelial cells (ECs), mesenchymal stem cells (MSCs), or any combination thereof. In some embodiments, the non-myocytes may comprise FBs in amount of about 50% to 60% based on the total number of non-myocytes, ECs in an amount of about 25% to about 35% based on the total number of non-myocytes, and MSCs in an amount of about 10% to about 20% based on the total number of non-myocytes. In some embodiments, the ECs may comprise human umbilical vein endothelial cells (HUVECs) and/or MSCs may comprise human adipose derived stem cells (hADSCs).

The cells can be homogenously mixed to a density of 1×10⁵, 1×10⁶, 1×10⁷, 1×10⁸, to 1×10⁹ cells/mL within an alginate-gelatin bioink mixture or similar hydrogel forming mixture. The hydrogel is permeable for dissolved oxygen and permeable for medium components and preferably permeable for metabolic products, e.g. permeable for diffusion. In certain aspects the hydrogel is an alginate-gelatin hydrogel or alternatively is based of an extracellular matrix of basal membranes of animal cells. The hydrogel can be on the basis of laminin and/or entactin and/or collagen and/or fibronectin and/or PEG, optionally on the basis of PEG (polyethylene glycol), e.g. in combination with fibronectin. The hydrogel preferably has a gelatinous structure. An alternative hydrogel is Matrigel. Mixing can be performed using 2 luer lock syringes or other mixing methods to make the cell density homogenous throughout the mixture. The cell-gel mixture can be loaded into an SLA printed cassette having micropillars forming a cell culture chamber. The cell culture chamber coupled to or within a bioreactor. The culture chamber being rotated at 5, 10, 15, 20, 30, 35, 40, to 45 rpm under appropriate culture conditions (e.g., 5% CO₂, 37° C.) for five days.

This microfluidic device/cell culture chamber described herein maintains continuous fluid circulation and laminar flow within the chamber. The micropillars of the microfluidic device serve as biophysical cues to help align cells to form organoids in self-assembled units and assist with continuously moving liquid through the device. Precise parameters such as fluid inlet velocities and viscosity, channel dimensions, effective mass transfer rates of nutrients and metabolic wastes, dynamic flow rate for matching the growth and remodeling of the organoids in relation to optimization of cell growth conditions was achieved by using the Navier-Stokes equation.

Other embodiments are directed to three-dimensional (3D) bioprinted scaffolds of hydrogel (e.g., gelatin-alginate) constructs encapsulated with a mixture of cells. In one aspect the cell mixture is, for example human cardiac AC16 cardiomyocytes (AC16-CMs), CFs, and microvascular ECs as cardiac organoid model. Studies showed heterocellular coupling normally exhibited between AC16-CM and CF by the expression of FSP-1 by the CF and CX43 by the CM. Due to the introduction of a third cell type, all the cells were expected to communicate with each other through direct cell-cell interactions (CM-CF) and paracrine signaling (between EC and other cells), as both homotypic and heterotypic cell interactions, which contribute to the organized structure and proper function of the heart. Direct communication was observed between all of the cells, CMs, CFs, and ECs, in the bioprinted scaffold, after 10-11 days of culture. The extent of the number of cell coupling improved with time and was significantly greater at 10-11 days, which continued to increase, reaching a peak at 20-21 days.

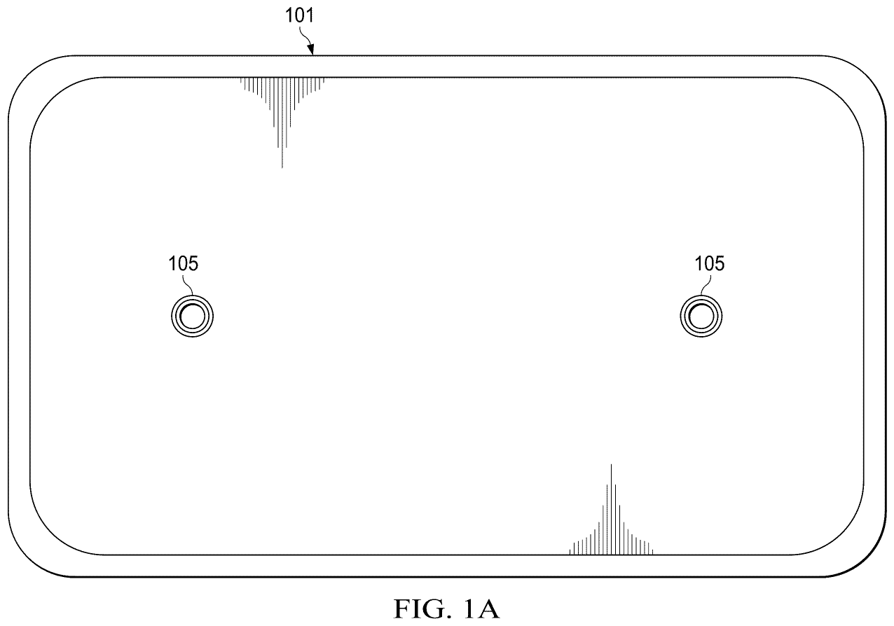

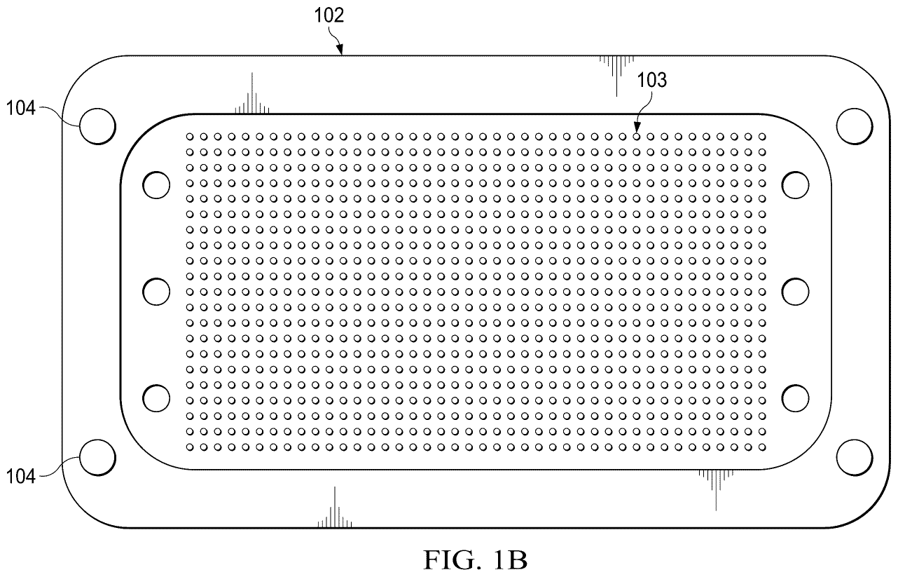

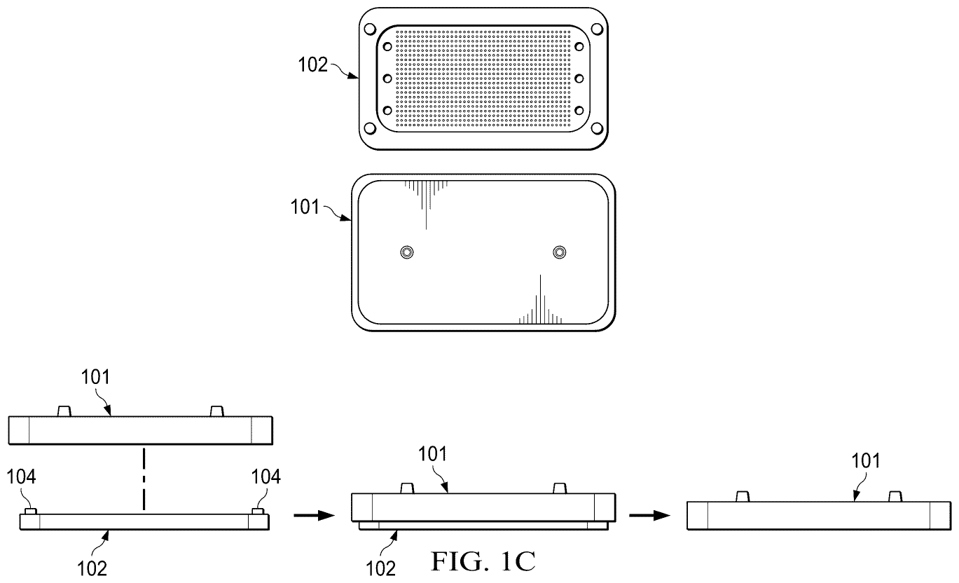

Schematic illustrations of an example of a microfluidic chip with a micropillar array are shown in FIGS. 1A-1C. FIG. 1A is a top view of the first layer (lid 101) with ports 105, FIG. 1B is a top view of the second layer (support/base 102) with a micropillar array 103, and FIG. 1C is a top and side view of both layers assembled. The microfluidic chip was tested using dyed water. Green food dye (˜300 μL) was added to the cell chamber. The lid was placed directly on top of the cell chamber and locked into place using the locking pins 104. The entire device was placed into the bioreactor with 5 ml of water and rotated at maximum speed. The device was left in the bioreactor for 48 hours. After 10 minutes, the water and food dye started mixing evenly. After 48 hrs, the food dye and water were mixed.

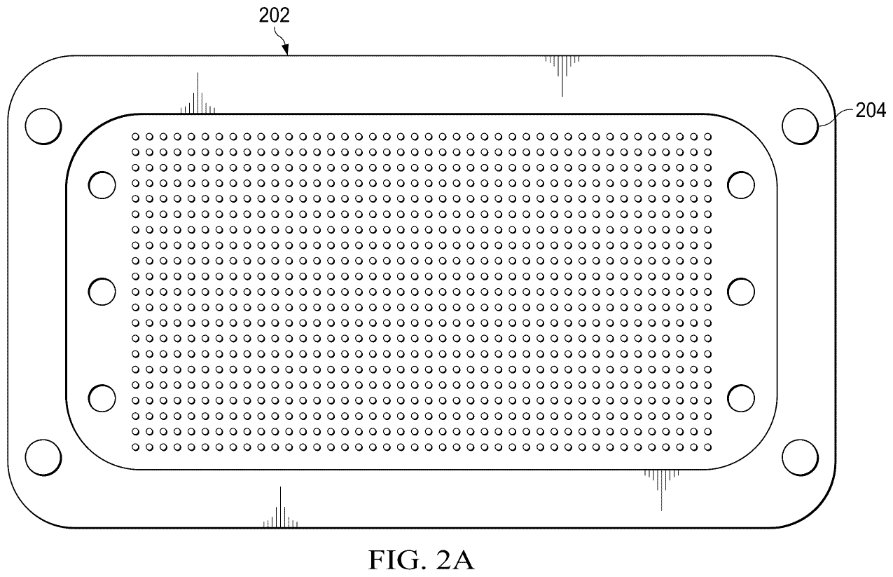

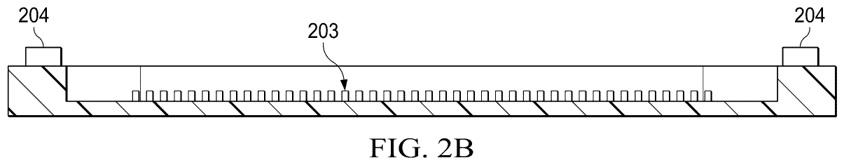

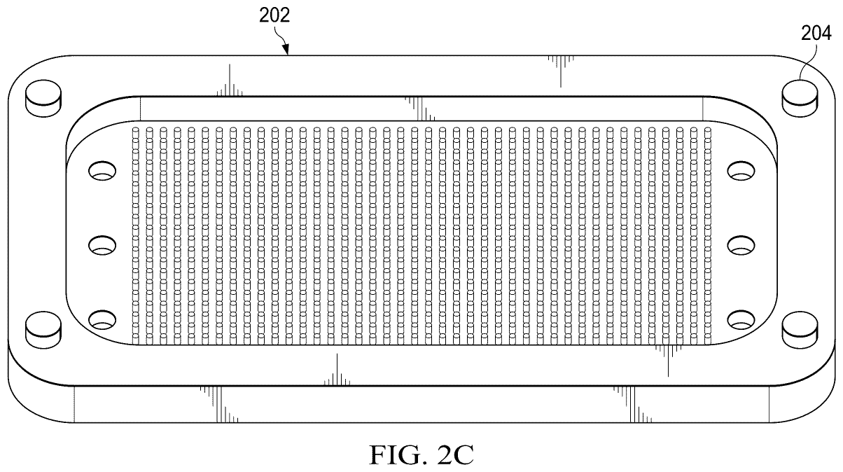

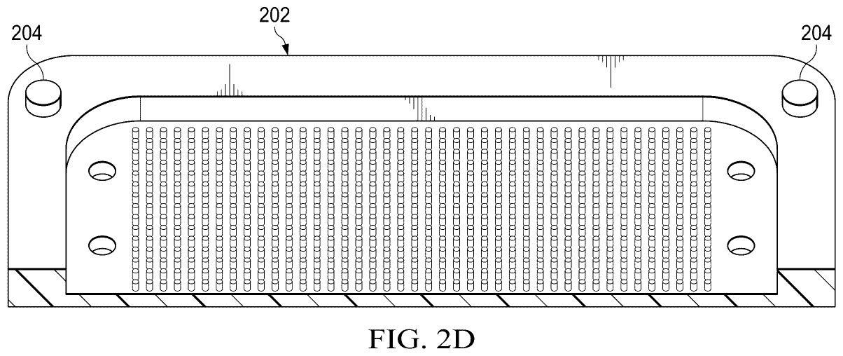

FIGS. 2A-2D illustrate various views of a cell culture chamber having a micropillar array. FIG. 2A is illustrating a top view of a base 202 having micropillars 203 and locking pins 204, FIG. 2B is illustrating a cross-section, FIG. 2C is illustrating a perspective view and FIG. 2D is a perspective view cross-section illustrating the micropillars of the second layer or cell chamber base.

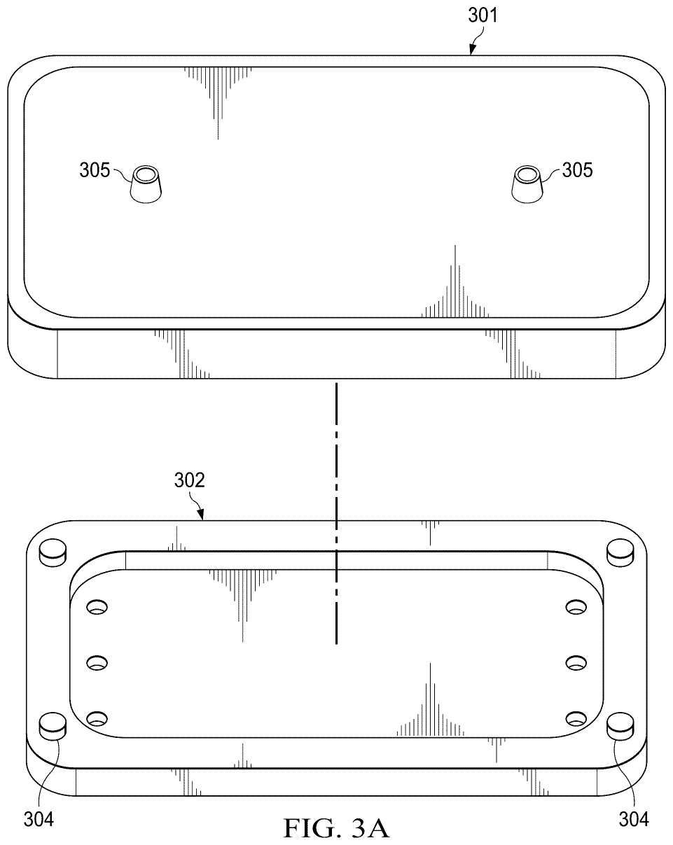

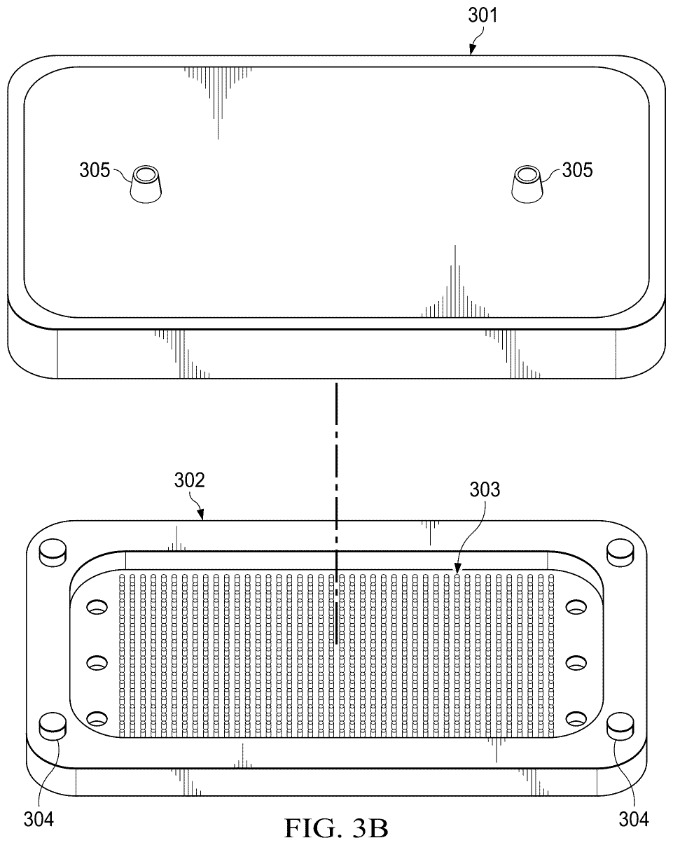

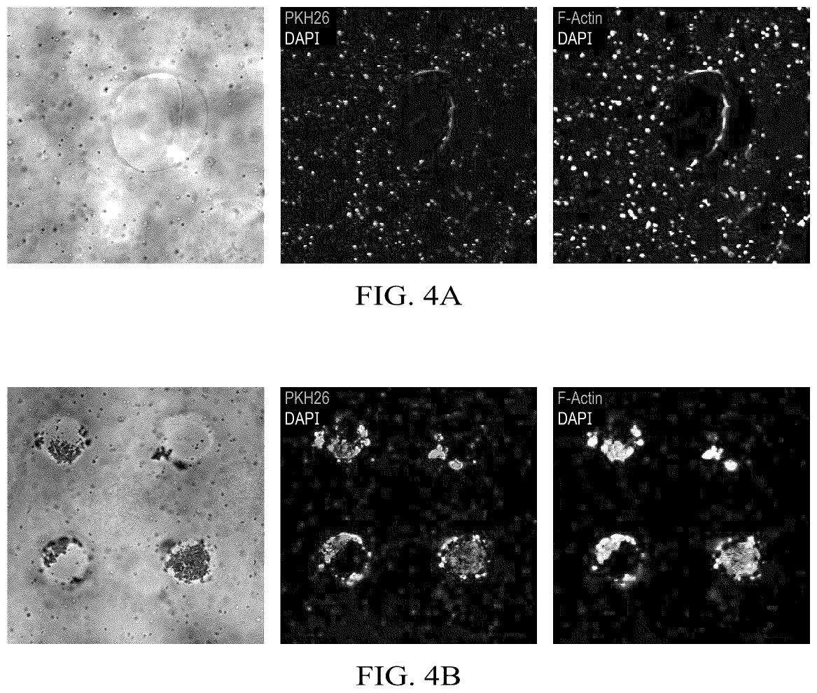

FIG. 3A illustrates a perspective view of an assembly of a non-pillared cell chamber having lid 301 with ports 305, base 302 with locking pins 304. FIG. 3B illustrates a perspective view of an assembly of a pillared cell chamber having lid 301 with ports 305, base 302 with locking pins 304 and micropillar array 303.

Preliminary tests were conducted with hydrogel. Two complete 3D printed devices were soaked in 70% EtOH for 10 minutes and placed under UV light for 10 minutes for sterilization. AC16 CM cells at density of 1.5×10⁶ cells/mL were cultured and split, stained with PKH26 red dye, and added to gelatin alginate hydrogel cross-linked using CaCl₂). Both cell chambers were placed in a petri dish and the hydrogel and cell mixture was added to each cell chamber at equal volume. The petri dish was placed on a Belly Dancer Shaker to promote equal distribution of the hydrogel and cell mixture. Each lid was added to its respective cell chamber and locked into place. The two complete devices with the hydrogel and cell mixture were split into two 35 mm petri dish (one dynamic, and the other static). Media was added to submerge the device completely. Both petri dishes were placed into the incubator at standard culture conditions for 24 hours to allow the cells to attach to the hydrogel. After 24 hours, the device in the dynamic petri dish was removed and added into the bioreactor with 5 mL FBS and rotated at max speed. The dynamic sample is left for 5 days in the bioreactor. The static device remains in the incubator for 5 days at standard culture condition, completely submerged in media.

### I. Characterization of Organoid Models

Electrophysiology (EPHYS). Advancements in organoid models for neural and cardiac tissue have established the feasibility for the use of such a model for improved drug toxicity screening, disease pathogenesis, and other environmental simulations. Their use, combined with microfluidic devices and 3-dimensional cell culture, has only furthered the capabilities of these constructs in various applications by coming closer to mimicking the physiological responses of native tissues. Of these, cerebral organoid models have become the most widely studied due to easily identifiable electrical occurrences by which intercellular communication is carried out in neurons. Thus, giving rise to characterization via electrophysiological (EPHYS) techniques as a critical validation tool for use in any application. Neuronal lipid membranes are composed of mechanosensitive protein-gated channels that allow for the inward and outward flow of Na+, Ca+, K+, and Cl ions. The flux of which is mitigated by the presence of electrical potential in the cell membrane. In response to stimulation, neurons transmit electrical signals, known as action potentials, along the axon to allow for the influx or release of ions. Action potentials caused by neuronal firing can then be observed via EPHYS techniques. Monitoring the electrophysiological responses produced by organoids has proven to be a useful indicator of the presence of a healthy, functioning cellular network. Perhaps the most distinguishing feature of tissue-on-a-chip models is the culture of cells in a 3-D environment rather than 2-D. This array allows for enhanced cell-cell communications, thus improving cellular activity as model conditions approach those observed in vivo. By this method, it is possible to achieve an arrangement in which there exist neighboring cells in x, y, and z directions for any given cell. This multi-directional cellular communication is imperative to developing ‘normal functioning’ networks. Successes and continual optimization of EPHYS characterization for neuronal models has incited a shift towards the use of such techniques in cardiac models. Like neurons, cardiac cells possess a negative resting membrane potential that changes due to the outward flow of K+ ions, which can be measured as electrical current. Though resting and stimulated potentials vary slightly from neuronal cells, it is understood that the same EPHYS techniques and organoid optimization concepts can easily be translated to cardiac organoids.

Patch-Clamp, Microelectrode Arrays (MEA). Previous electrophysiology work relies on a traditional whole-cell recording technique known as patch-clamp. In this process, a micropipette tip connected to a recording electrode is placed in contact with a targeted cell. Suction is applied to create a seal between the cell membrane and pipette tip, thus isolating the ion channels in that region. The cell is stimulated via induced current or pharmaceutical delivery and the ion response is recorded in terms of measured voltage across the cell membrane. This provides valuable insight into the electrophysiological properties of cells in response to intentional manipulation. However, this technique allows only for the observation of an individual cell's response while also imposing damage to the cell membrane. Consequently, this method is supplemented with calcium imaging to corroborate cell-cell communication within the network. In one study, which utilized human iPSC-CMs derived from patients with autosomal polycystic kidney disease to study the development of cardiovascular disease which is often associated with the disorder. Calcium imaging revealed decreased sarcoplasmic reticulum content in the diseased cell types along with decreased PKD1 gene types. Whole-cell patch clamp recording was then utilized to assess L-type calcium currents, baseline action potentials, and drug responsiveness of diseased cells in comparison to control cells, enabling researchers to demonstrate proof of concept for the utilization of patient-specific iPSC models for human diseases (Lee et al., Stem Cell Res 25:83-7, 2017). As can be seen, the use of calcium imaging and patch-clamp electrophysiology in parallel has proven to be an effective tool for identifying formations and relative function of cellular networks in organoid models. Still, the limitations imposed by this technique offer no insight into network function as a whole, which has led recent studies to shift toward the use of microelectrode arrays (MEA) for the characterization of organoid function. MEAs are chips including multiple microscopic electrodes which are used to collect extracellular EPHYS data from a wide number of cells in a population. This provides significantly more data compared to patch-clamp technique, which utilizes only a single electrode and measures activity from a single cell, thus creating a higher-throughput model for organoid network characterization. One study successfully recorded both the spontaneous and evoked response electrical activity of hiPSC-derived neurons plated on a biochip coated with PDLO for up to 3 months (Amin et al., Frontiers in Neurosci 10:121, 2016). This approach demonstrates the use of such devices for long-term observation of networks of cell cultures by multi-site data acquisition which could not have been obtained with traditional methods. Though early works focused on recording data from a 2D culture grown over the top of multi electrode arrays, increasing potential in applications for organoid models has led to the combination of this technology with 3D cultures maintained in microfluidic devices. Advancements in technology have also made possible the fabrication of MEAs in various arrays designed for specific applications such as those embedded in well plates, probe, and mesh-types to be embedded throughout the organoid.

RNA-sequencing. Changes in global gene expression profiles due to processing via various methods for organoid formation can be accessed via RNA-sequencing (RNA-seq), a high throughput-sequencing assay that can provide an unbiased quantification of all expressed genes in the engineered cardiac tissue-on-a-chip platform and will also allow identification of the key cellular signaling pathways that are perturbed, in response to environmental stressors. Generalized linear models (GLMs) can be adopted to identify differentially expressed genes before and after exposure. This can be a useful tool to reveal cell damage pre- and post-exposure to environmental stressors. Analysis of transcriptome dynamics will also allow identification of the key cellular signaling pathways that may be perturbed by long term culture of the cardiac organoids. This data can be leveraged to assess the impact of heterocellular culture on aging/senescence or trans-differentiation. For downstream functional analysis, Gene Ontology (GO) enrichment and Gene Set Enrichment Analysis (GSEA) analysis can be performed on the differentially expressed genes.

Single-cell RNA sequencing (scRNA-seq) provides an in-depth analysis of complex cell populations, cell lineages, as well as the relation between different genes. This technique solves the limitations of bulk RNA-sequencing since scRNA-seq does not provide the average gene expression among multiple cells. ScRNA-seq allows scientists to obtain an unbiased analysis of cellular heterogeneity. The implementation of these technologies has been critical in the development and implementation of 3D cardiac organoids. Additionally, scientists have developed a different range of single-cell methods to provide useful data. The workflow to perform scRNA-seq includes sample preparation, single-cell capture, reverse transcription, amplification, library preparation, sequencing, and analysis. For this technique, Differential Expression (DE) analysis and Gene Set Enrichment (GSE) analysis are most commonly used. DE identifies the different genes expressed in cell subpopulations as well as comparing gene expression between experimental conditions and case control samples, this analysis uses methods such as MAST, SCDE, and zingeR, which are used to analyze one gene at a time. Likewise, GSE provides the significant difference between two distinct biological states, this analysis utilizes multiple gene set analysis methods such as Significance Analysis of Function and Expression (SAFE) and Correlation Adjusted Mean Rank (CAMERA).

A single-cell approach is especially useful for cardiac applications due to the different cell types found in the heart. In previously published work scientists have implemented the use of RNA-sequencing to validate the mimicking characteristics of 3D cardiac organoids in relation to the human body. Rossi et al. developed a multicellular cardiac organoid in which they implemented a scRNA-seq analysis, which resulted in gene expression sequences containing MESP1, RYR2, and alpha-actinin, which are markers of cardiac progenitor cells and mature cardiomyocytes.

Imaging and algorithm-based analysis. The development of 3D cardiac organoids and advanced imaging techniques to characterize these models necessitates a high-throughput, automated image analysis and quantification process. Current image analysis algorithms are primarily written in Python or Matlab code and incorporate established computer vision libraries, packages, and modules (collectively called tools or algorithms). These tools like skimage-watershed and scipy-ndimage implement image analysis functions such as segmentation, sharpening, de-speckle, rotation/translation, and pixel intensity detection to identify and characterize objects of interest. The quantitative output of this analysis includes for these objects includes pixel intensity values (correlated to fluorescence intensity), automated counts per image, mean distance to nearest neighbor, and size/shape measurements. The basic workflow to incorporate ML image analysis in organoid characterization is: (1) immunofluorescence staining (2) 3D fluorescence/confocal microscopy to acquire z-stack images (3) organize/process z-stack images to desired quality with ImageJ or similar software, and (4) apply ML algorithm for automated image analysis.

Toepfer et al. (Circ Res 124:1172-83, 2019) developed a MATLAB software for large-scale analysis of sarcomere function in human induced pluripotent stem cell derived cardiomyocytes (hiPSC-CMs). This program, SarcTrack, was developed to monitor sarcomere count and dynamic changes in sarcomere length (SL), parameters for sarcomeres, percent and sarcomere contraction, relaxation durations, and cellular beat rate. These measures were obtained using fluorescent videos of labeled Z-disc or M-line pairs within each sarcomere. hiPSC-CM sarcomere proteins were fluorescently tagged using CRISPR/Cas9-mediated homology-directed repair of hiPSCs to introduce red fluorescent protein (RFP) onto the carboxyl terminus of Myomesin-1 (MYOM1-RFP), which is an M-band protein. For imaging, hiPSC-CMs were replated and imaged at day 30 after post differentiation. Fluorescent tags were delivered by mApple-ACTN-2 lentivirus. Contractile forces were exerted by hiPSC-CMs on micropatterned fibronectin polyacrylamide hydrogel substrates at 1 Hz and videos of bead motion near the substrate surface were acquired at 30 frames per second. Five second video imaging on small hiPSC-CM clusters of 2_4 cells using a 100× objective of a fluorescent microscope at 30 frames per second. After removing sarcomeres of low fluorescence intensity, SarcTrack was able to detect real-time distances between sarcomere domains. To detect the duration of contraction and relaxation, individual sarcomeres were fitted to a period curve that could be custom designed. SarcTrack was compared to synthetic computer-generated sarcomeres. Here, Z-discs were generated with known parameters. Using 2 simulation contractions, SarcTrack was used to measure the synthetic sarcomeres and measured the prescribed values as identified. To assess contractility affected by fluorescent tags, SarcTrack analyzed GFP<RFP< and ACTN-2-mApple, showing comparable contractility and relaxation durations. There was little variance across replicates for sarcomere shortening, resting SLs, contraction durations, and relaxation durations. Contractile parameters were quantified over differentiation days, showing trends such as sarcomere shortening increased at day 12 and reached a plateau by day 20. Contractile cycle parameters where quantified, revealing beat rate had a substantial impact on contractility parameters. In addition, parallel analyses with common cardiac drugs, such as Propranolol, showed effects on sarcomere performance while Verapamil, had minimal effects on duration of contraction and relaxation. SarcTrack demonstrated accuracy and functional assessment of sarcomeres in hiPSC-CMs. However, SarcTrack needed to account for fluorescent labeled, paired domains of sarcomeres in cells without high background signal. Input videos into SarcTrack must be 30 frames per second or more with good signal-to-noise ratio, which may limit the types of fluorescence confocal microscopes that cannot obtain high frame rate videos. SarcTrack demonstrated the ability to evaluate sarcomere contractile function with a set of 6 parameters and in turn fitted to an individual sarcomere in a frame, an application that can be used to address sarcomere analysis platforms.

Recently a cursory ML analysis was conducted on a small set of images collected during ongoing work developing cardiac organoids in dynamic microfluidic culture systems. Customized image processing pipelines were implemented using computer vision techniques for approximating cell count, distribution, and stain absorption to quantify the extent of cardiac organoid formation. Automated image analysis has been used to address most of these quantitative measurements (Strobel et al. Frontiers in Physiol 12, 2021). The quantification process begins with preprocessing, where all z-stacked fluorescent images are scaled to the same magnification level, standardizing pixels per micron. Next, each image is converted to grayscale and threshold using Otsu's method (Otsu, IEEE transac on systems, man, and cybernetics 9:62-6, 1979), for computing the Otsu threshold for each image and using the lowest observed threshold across the set as the standard.

Noise was eliminated from the thresholded images with an erosion operation, and then computed a distance transform map, which calculates the distance of each pixel to the nearest zero-valued pixel. The local peaks of the distance transformed images indicated the nominal centers of convex objects and were used as seed points in a watershed segmentation (Vincent and Soille, IEEE Transac Pattern Analysis & Machine Intelligence 13:583-98, 1991). The resulting segmentation yields instances of round image objects, corresponding to detected cells or clusters of cells. With cell clusters identified in comparison with no clusters, several quantitative metrics can be computed, including counts, densities, pixel brightness/intensity distributions, and cluster sizes. Without the presence of posts, the cluster intensities are greater due to the homogenous spreading of cells throughout the culture platform.

In summary, a very simple processing pipeline can extract meaningful analytics for images of this type, enabling output of quantitative data. Notably, this processing pipeline requires no upfront manual annotation of images. While computer vision is more broadly approachable using deep learning techniques, this particular problem is better approached with conventional processing as there is no need for the large receptive field of a neural network, and the quantity of interest (intensity) is being measured directly.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

1. A cardiac organoid platform comprising an induction support and an induction media, (i) the induction support comprising micropillars extending from a base of the induction support and (ii) the induction media comprising a hydrogel support and at least one of a cardiomyocyte or a cardiomyocyte precursor.

2. The cardiac organoid platform of claim 1, wherein micropillars are 0.1 mm to 1 mm in height and 100 μm to 1 mm in diameter.

3. The cardiac organoid platform of claim 2, wherein the micropillars are spaced at a density of 100 to 10,000 pillars per mm².

4. The cardiac organoid platform of claim 1, wherein the hydrogel support comprises an alginate hydrogel.

5. The cardiac organoid platform of claim 4, wherein the alginate hydrogel comprises 2 to 80 wt % alginate.

6. The cardiac organoid platform of claim 4, wherein the alginate hydrogel comprises an alginate-gelatin hydrogel.

7. The cardiac organoid platform of claim 6, wherein the alginate hydrogel comprises 2 to 80 wt % alginate.

8. The cardiac organoid platform of claim 1, wherein the cardiomyocyte comprises a pluripotent stem cell-derived cardiomyocyte.

9. The cardiac organoid platform of claim 8, wherein the pluripotent stem cell-derived cardiomyocyte comprises a patient-specific pluripotent stem cell-derived cardiomyocyte.

10. The cardiac organoid platform of claim 1, wherein the induction support comprises a lid, the lid and the base forming a cell culture chamber when in use.

11. A cardiac organoid culture system comprising the cardiac organoid platform of claim 1 and a rotation mechanism configured to rotate the cardiac organoid platform during culture.

12. A method of inducing organoid formation comprising:
encapsulating an induction support having an array of micropillars forming an encapsulated induction support;
culturing the encapsulated induction support in a cell culture chamber while rotating; and
perfusing the encapsulated induction template.

13. The method of claim 12, wherein the encapsulated induction support is cultured in a culture medium at a temperature of 35 to 40° C. and 2 to 8% CO₂ atmosphere.

14. The method of claim 12, wherein the encapsulated induction support is rotated at 20 to 60 rpms during culture.

15. A cardiac organoid platform, comprising:
an induction support; and
an induction media,
wherein the induction support comprises micropillars extending from a base of the induction support and defining an array,
wherein the induction media comprises a hydrogel support and at least one of a cardiomyocyte or a cardiomyocyte precursor,
wherein the hydrogel support comprises an alginate hydrogel, and
wherein the cardiomyocyte comprises a pluripotent stem cell-derived cardiomyocyte.

16. The cardiac organoid platform of claim 15, wherein micropillars are 0.1 mm to 1 mm in height and 100 μm to 1 mm in diameter.

17. The cardiac organoid platform of claim 16, wherein the micropillars are spaced at a density of 100 to 10,000 pillars per mm².

18. The cardiac organoid platform of claim 15, wherein the alginate hydrogel comprises 2 to 80 wt % alginate.

19. The cardiac organoid platform of claim 15, wherein the alginate hydrogel comprises an alginate-gelatin hydrogel.

20. The cardiac organoid platform of claim 19, wherein the alginate hydrogel comprises 2 to 80 wt % alginate.

21. The cardiac organoid platform of claim 15, wherein the pluripotent stem cell-derived cardiomyocyte comprises a patient-specific pluripotent stem cell-derived cardiomyocyte.

22. The cardiac organoid platform of claim 15, wherein the induction support comprises a lid, the lid and the base forming a cell culture chamber when in use.

23. A cardiac organoid culture system comprising the cardiac organoid platform of claim 15 and a rotation mechanism configured to rotate the cardiac organoid platform during culture.
